(19) United States
(12) Patent Application Publication
(10) Pub. No.: US 2004/0181254 A1
(43) Pub. Date: Sep. 16, 2004

(54) Sphygmomanometer cuff having double bladder

(76) Inventors: Tae-young Choi, Euiwang-shi (KR); Won-ky Kim, Seoul (KR)

Correspondence Address: SHLESINGER, ARKWRIGHT & GARVEY LLP, PATENT, TRADEMARK & COPYRIGHT LAW, 3000 SOUTH EADS STREET, ARLINGTON, VA, 22202, US

(21) Appl. No.: 10/657,705

(22) Filed: Sep. 9, 2003

(30) Foreign Application Priority Data
Mar. 14, 2003 (KR) 10-2003-0016004

(51) Int. Cl. A61B 17/00

(52) U.S. Cl. 606/202

(57) ABSTRACT

Disclosed is a sphygmomanometer cuff having a double bladder. The sphygmomanometer cuff comprises an envelope placed around a desired portion of the human body such as the arm, wrist, finger, leg, or the like and functioning to protect bladders; a large bladder installed in the envelope to be inflated when air is supplied therein; and a small bladder installed in the envelope in conjunction with the large bladder to be inflated when air is supplied therein and effectively apply pressure to the artery extending through the arm, wrist, finger, leg, or the like, of the human body.

### CROSS-REFERENCE TO RELATED APPLICATION

This application claims the priority of Korean Application No. 10-2003-0016004, filed Mar. 14, 2003, which is incorporated herein by reference.

1. Field of the Invention

The present invention relates, in general, to a sphygmomanometer cuff and, more particularly, to a sphygmomanometer cuff having a double bladder, in which a small bladder is provided in addition to a large bladder to allow relatively large pulse waves to pass through the small bladder, thereby being capable of precisely and finely measuring a systolic blood pressure and a diastolic blood pressure through the pulse waves transmitted from the artery of the human body.

2. Background of the Invention

As well known to those skilled in the art, blood pressure is an important medical indicator due to the fact that the blood pressure is used as a standard for judging a condition of one's health. A sphygmomanometer is employed to measure blood pressure.

The sphygmomanometer utilizes a cuff for applying pressure to a desired portion of the human body such as the arm, wrist, finger, leg, or the like and then sensing pulse waves. As can be readily seen from FIG. 1, the conventional sphygmomanometer cuff comprises an envelope 1 which is placed around a portion of a subject's body and protects a bladder 2, the bladder 2 installed in the envelope 1 to be inflated when air is supplied therein, and a hose 3 connected to the bladder 2 to allow air to be supplied into or removed from the bladder 2.

Referring to FIG. 2, when it is necessary to use the conventional sphygmomanometer cuff constructed as mentioned above, the envelope 1 is wrapped around the arm, wrist, finger, leg, or the like of the subject's body, and then, air is pumped into the bladder 2 installed in the envelope 1. Thereupon, as the bladder 2 is inflated with the air, the entire cuff applies pressure to the arm, wrist, finger, leg, or the like so as to block the blood flow. Thereafter, by slowly deflating the pressurized air pumped into the bladder 2 through the hose 3, blood flows again through the artery of the arm, wrist, finger, leg, or the like of the subject's body. As a consequence, as pulse waves corresponding to the blood flow are transmitted to a sphygmomanometer, blood pressure can be measured.

However, the conventional sphygmomanometer cuff suffers from defects in that changes in pulse wave, which indicate a systolic blood pressure at the moment blood initially passes through the artery of the subject after discharge of air from the cuff is started, and a diastolic blood pressure at the moment blood passes without resistance through the artery, are not clearly and distinctly transmitted to the bladder. As a result, it is difficult to precisely measure the systolic and diastolic blood pressures using the conventional sphygmomanometer cuff.

### SUMMARY OF THE INVENTION

Accordingly, the present invention has been made keeping in mind the above problems occurring in the prior art, and an object of the present invention is to provide a sphygmomanometer cuff having a double bladder, in which a small bladder of a predetermined size is provided in addition to a large bladder at a desired position to allow relatively large pulse waves to be transmitted to the small bladder, thereby being capable of generating pulse waves for allowing a systolic blood pressure at the moment blood initially passes through the artery of the subject's body after deflating of air from the cuff is started, and a diastolic blood pressure at the moment blood passes without resistance through the artery, to be effectively measured.

In order to achieve the above object, according to the present invention, there is provided a sphygmomanometer cuff having a double bladder, comprising: an envelope placed around a desired portion of the human body such as the arm, wrist, finger, leg, or the like and functioning to protect bladders; a large bladder installed in the envelope to be inflated when air is supplied therein; and a small bladder installed in the envelope in conjunction with the large bladder to be inflated when air is supplied therein and effectively apply pressure to the artery extending through the arm, wrist, finger, leg, or the like, of the human body.

### BRIEF DESCRIPTION OF THE DRAWINGS

The above and other objects, features and other advantages of the present invention will be more clearly understood from the following detailed description when taken in conjunction with the accompanying drawings, in which:

FIG. 1 is a constructional view illustrating a conventional sphygmomanometer cuff;

FIG. 2 is a sectional view illustrating an in-use status of the conventional sphygmomanometer cuff;

FIG. 3 is a sectional view illustrating an in-use status of a sphygmomanometer cuff having a double bladder in accordance with a first embodiment of the present invention;

FIG. 4 is a constructional view illustrating a sphygmomanometer cuff having a double bladder in accordance with a second embodiment of the present invention;

FIG. 5 is a sectional view taken along the line A-A of FIG. 4;

FIG. 6 is a sectional view illustrating a variation of FIG. 5;

FIG. 7 is a constructional view illustrating a sphygmomanometer cuff having a double bladder in accordance with a third embodiment of the present invention;

FIG. 8 is a sectional view taken along the line B-B of FIG. 7;

FIG. 9 is a sectional view illustrating a variation of FIG. 8;

FIG. 10 is a constructional view illustrating a sphygmomanometer cuff having a double bladder in accordance with a fourth embodiment of the present invention;

FIG. 11 is a sectional view taken along the line C-C of FIG. 10;

FIG. 12 is a sectional view illustrating a variation of FIG. 11;

FIG. 13 is a constructional view illustrating a sphygmomanometer cuff having a double bladder in accordance with a fifth embodiment of the present invention.

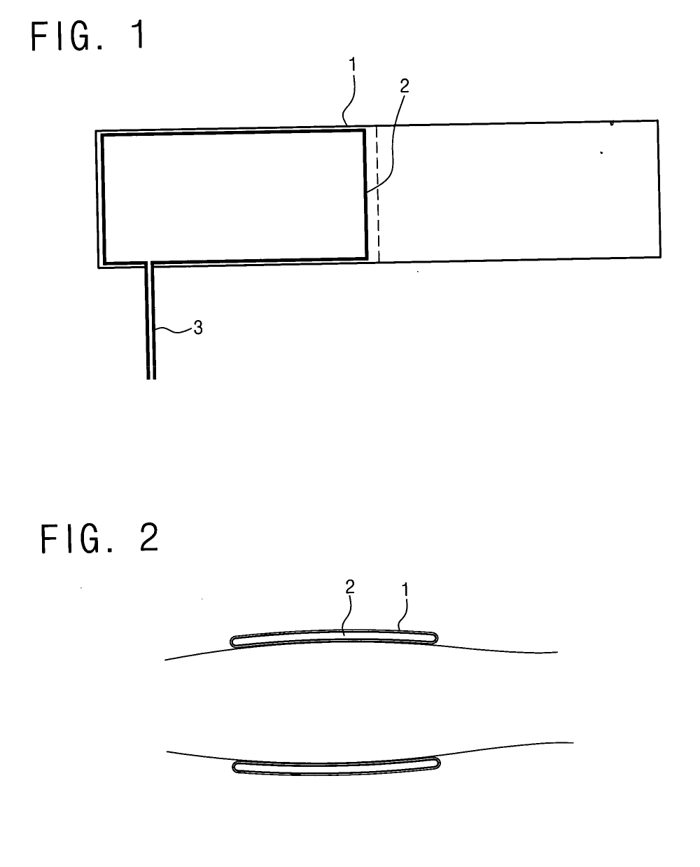

### DETAILED DESCRIPTION OF THE INVENTION

Reference should now be made to the drawings, in which the same reference numerals are used throughout the different drawings to designate the same or similar components.

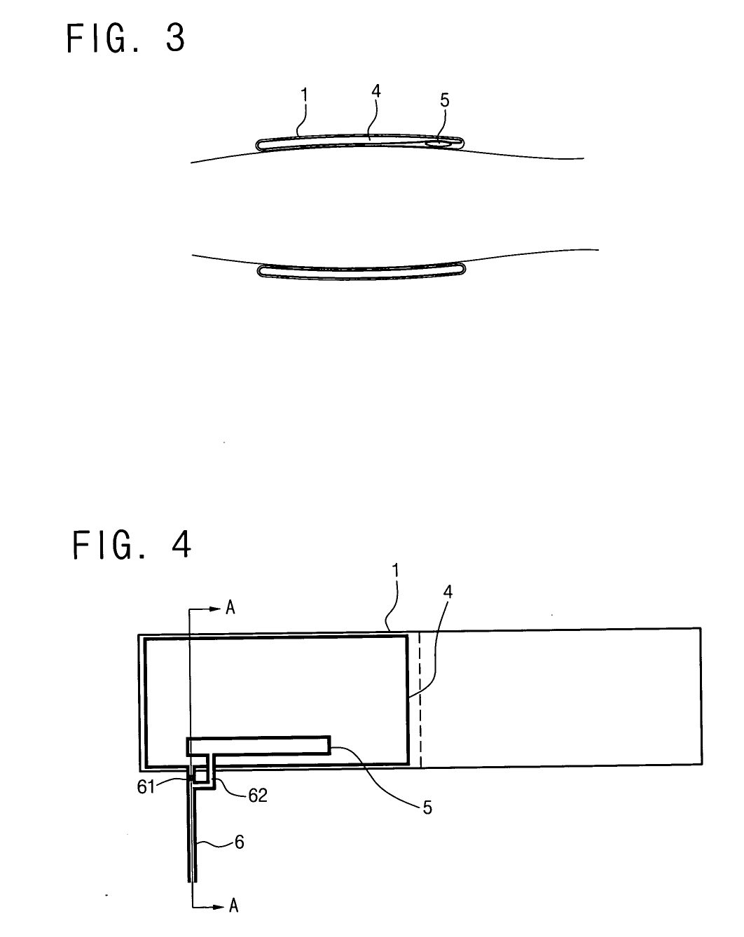

A construction of a sphygmomanometer cuff having a double bladder in accordance with a first embodiment of the present invention comprises, as shown in FIG. 3, an envelope 1 which is placed around a portion of the human body and functions to protect bladders, a large bladder 4 which is installed in the envelope 1 to be inflated when air is supplied therein, and a small bladder 5 which is installed in the envelope 1 in conjunction with the large bladder 4 to be inflated when air is supplied therein and apply pressure to the artery extending through the arm, wrist, finger, leg, or the like, in cooperation with the large bladder 4.

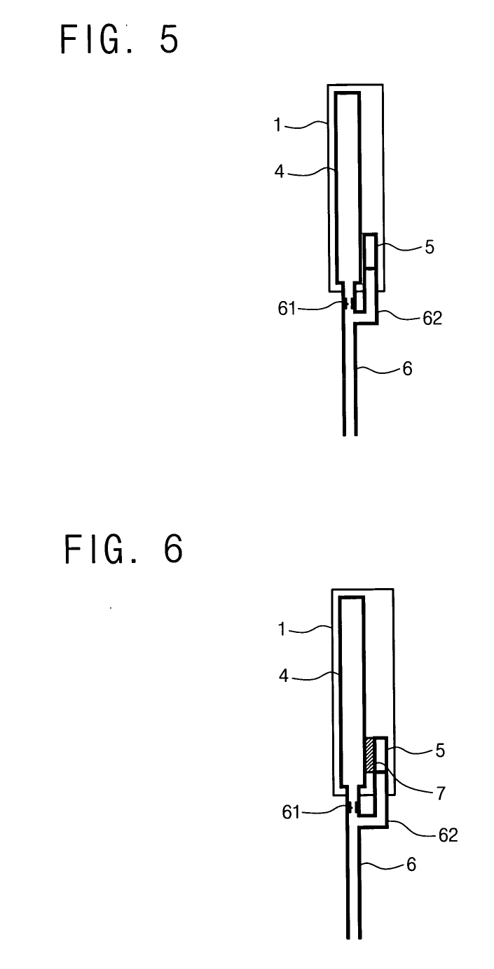

A construction of a sphygmomanometer cuff having a double bladder in accordance with a second embodiment of the present invention comprises, as shown in FIGS. 4 and 5, an envelope 1 which is placed around a portion of the subject's body and functions to protect bladders, a large bladder 4 which is installed in the envelope 1 to be inflated when air is supplied therein, and a small bladder 5 which is installed in the envelope 1 in conjunction with the large bladder 4 to be inflated when air is supplied therein and apply pressure to the artery extending through the arm, wrist, finger, leg, or the like, of the subject, in cooperation with the large bladder 4. In this second embodiment of the present invention, a hose 6, which has a first connection part 61 of a small diameter and a second connection part 62 of a large diameter, is provided in a manner such that the first connection part 61 is connected with the large bladder 4 and the second connection part 62 is connected with the small bladder 5.

A construction of a sphygmomanometer cuff having a double bladder in accordance with a variation of the second embodiment of the present invention further comprises, as shown in FIG. 6, a spacer 7 which is interposed between the large and small bladders 4 and 5. At this time, it is preferred that the spacer 7 is bonded between the large and small bladders 4 and 5 by an adhesive.

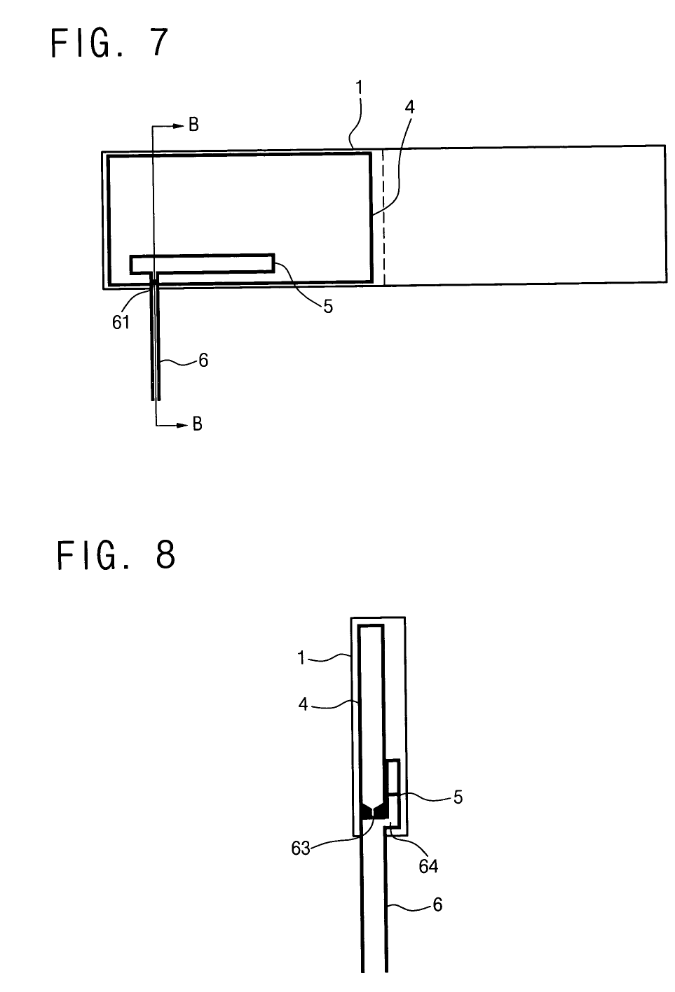

A construction of a sphygmomanometer cuff having a double bladder in accordance with a third embodiment of the present invention comprises, as shown in FIGS. 7 and 8, an envelope 1 which is placed around a portion of the subject's body and functions to protect bladders, a large bladder 4 which is installed in the envelope 1 to be inflated when air is supplied therein, and a small bladder 5 which is installed in the envelope 1 in conjunction with the large bladder 4 to be inflated when air is supplied therein and apply pressure to the artery extending through the arm, wrist, finger, leg, or the like, of the subject, in cooperation with the large bladder 4. In this third embodiment of the present invention, after a first connection part 63 of a small diameter is connected with the large bladder 4 and a second connection part 64 of a large diameter is connected with the small bladder 5, the first and second connection parts 63 and 64 are connected to a hose 6.

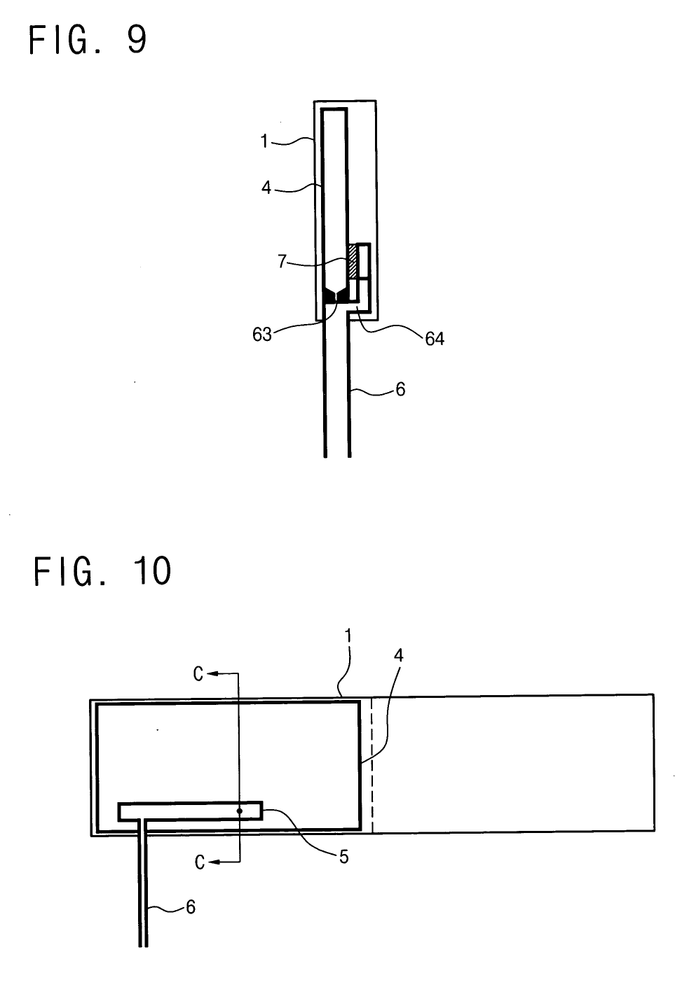

A construction of a sphygmomanometer cuff having a double bladder in accordance with a variation of the third embodiment of the present invention further comprises, as shown in FIG. 9, a spacer 7 which is interposed between the large and small bladders 4 and 5. At this time, it is preferred that the spacer 7 is bonded between the large and small bladders 4 and 5 by an adhesive.

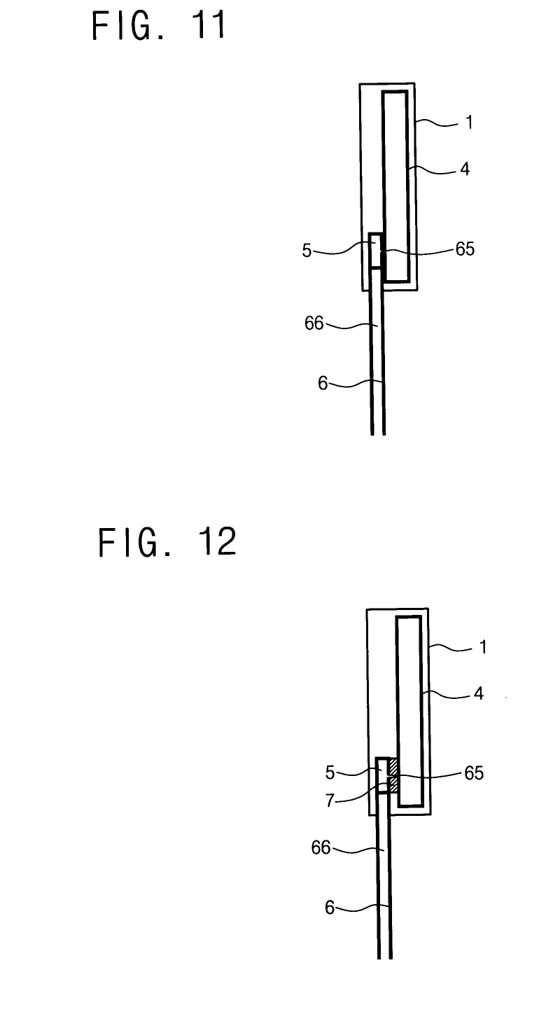

A construction of a sphygmomanometer cuff having a double bladder in accordance with a fourth embodiment of the present invention comprises, as shown in FIGS. 10 and 11, an envelope 1 which is placed around a portion of the subject's body and functions to protect bladders, a large bladder, 4 which is installed in the envelope 1 to be inflated when air is supplied therein, and a small bladder 5 which is installed in the envelope 1 in conjunction with the large bladder 4 to be inflated when air is supplied therein and apply pressure to the artery extending through the arm, wrist, finger, leg, or the like, of the subject, in cooperation with the large bladder 4. In this fourth embodiment of the present invention, after a first connection part 65 of a small diameter is installed between the large bladder 4 and the small bladder 5 and a second connection part 66 of a large diameter is installed on the small bladder 5, the second connection part 66 of the large diameter is connected to a hose 6.

A construction of a sphygmomanometer cuff having a double bladder in accordance with a variation of the fourth embodiment of the present invention further comprises, as shown in FIG. 12, a spacer 7 which is interposed between the large and small bladders 4 and 5. At this time, it is preferred that the spacer 7 is bonded between the large and small bladders 4 and 5 by an adhesive.

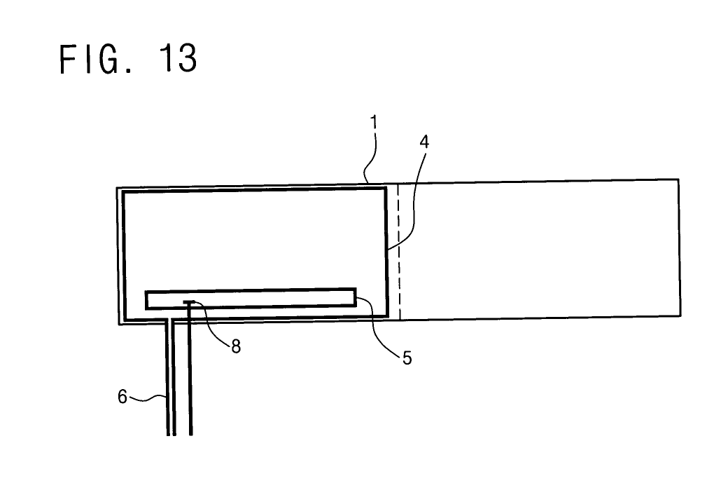

A construction of a sphygmomanometer cuff having a double bladder in accordance with a fifth embodiment of the present invention comprises, as shown in FIG. 13, an envelope 1 which is placed around a portion of the subject's body and functions to protect bladders, a large bladder 4 which is installed in the envelope 1 to be inflated when air is supplied therein, and a small bladder 5 which is installed in the envelope 1 in conjunction with the large bladder 4 to be inflated when air is supplied therein and apply pressure to the artery extending through the arm, wrist, finger, leg, or the like, of the subject, in cooperation with the large bladder 4. In this fifth embodiment of the present invention, the large bladder 4 is connected to a hose 6, and a sensor 8 is airtightly installed in the small bladder 5.

Hereinafter, operations of the sphygmomanometer cuff according to the present invention, constructed as mentioned above, will be described.

After the envelope 1 is wrapped around the arm, wrist, finger, leg, or the like of the subject, air is pumped through the hose 6 into the large and small bladders 4 and 5 which are installed in the envelope 1. Thereupon, as the large and small bladders 4 and 5 are inflated with air, the entire cuff applies pressure to the artery of the arm, wrist, finger, leg, or the like of the subject so as to block the blood flow.

Thereafter, by slowly deflating through the hose 6 the pressurized air pumped into the large and small bladders 4 and 5, pulse waves are transmitted from the artery of the subject to the large and small bladders 4 and 5, whereby the pulse waves can be reliably sensed by a sphygmomanometer.

In the present invention, the small bladder 5 has a volume which is significantly less than that of the large bladder 4. When viewed in a blood flowing direction, the small bladder 5 is positioned at the center of or at the back of the large bladder 4. Due to the fact that the second connection parts 62, 64 and 66 for connecting the large bladder 4 with the hose 6 have diameters which are larger than those of the first connection parts 61, 63 and 65 for connecting the small bladder 5 with the hose 6, while an air pressure in the cuff is gradually decreased, at the moment that blood starts to flow through the artery, a large pulse wave is generated particularly in the small bladder 5, whereby systolic and diastolic blood pressures can be measured in a precise and easy manner.

As apparent from the above description, the sphygmomanometer cuff having a double bladder according to the present invention, constructed as mentioned above, provides advantages in that a small bladder is provided in addition to a large bladder to allow relatively large pulse waves to be transmitted to the small bladder, whereby it is possible to precisely measure a systolic blood pressure at the moment blood initially passes through the artery of the subject after deflating of air supplied into the cuff is started and a diastolic blood pressure at the moment blood passes without resistance through the artery of the human body.

Although a preferred embodiment of the present invention has been described for illustrative purposes, those skilled in the art will appreciate that various modifications, additions and substitutions are possible, without departing from the scope and spirit of the invention as disclosed in the accompanying claims.

What is claimed is:

1. A sphygmomanometer cuff having a double bladder, comprising:
an envelope placed around a desired portion of the human body such as the arm, wrist, finger, leg, or the like and functioning to protect bladders;
a large bladder installed in the envelope to be inflated when air is supplied therein; and
a small bladder installed in the envelope in conjunction with the large bladder to be inflated when air is supplied therein and effectively apply pressure to the artery extending through the arm, wrist, finger, leg, or the like, of the human body,
wherein a hose having a first connection part of a small diameter and a second connection part of a large diameter is provided in a manner such that the first connection part is connected with the large bladder and the second connection part is connected with the small bladder.

2. The sphygmomanometer cuff as set forth in claim 1, wherein a spacer is interposed between the large and small bladders.

3. The sphygmomanometer cuff as set forth in claim 1, wherein, when viewed in a blood flowing direction, the small bladder is positioned at the center of or at the back of the large bladder.

4. A sphygmomanometer cuff having a double bladder, comprising:
an envelope placed around a desired portion of the human body such as the arm, wrist, finger, leg, or the like and functioning to protect bladders;
a large bladder installed in the envelope to be inflated when air is supplied therein; and
a small bladder installed in the envelope in conjunction with the large bladder to be inflated when air is supplied therein and effectively apply pressure to the artery extending through the arm, wrist, finger, leg, or the like, of the human body,
wherein, after the first connection part is connected with the large bladder and the second connection part is connected with the small bladder, the first and second connection parts are connected to the hose.

5. The sphygmomanometer cuff as set forth in claim 4, wherein a spacer is interposed between the large and small bladders.

6. The sphygmomanometer cuff as set forth in claim 4, wherein, when viewed in a blood flowing direction, the small bladder is positioned at the center of or at the back of the large bladder.

7. A sphygmomanometer cuff having a double bladder, comprising:
an envelope placed around a desired portion of the human body such as the arm, wrist, finger, leg, or the like and functioning to protect bladders;
a large bladder installed in the envelope to be inflated when air is supplied therein; and
a small bladder installed in the envelope in conjunction with the large bladder to be inflated when air is supplied therein and effectively apply pressure to the artery extending through the arm, wrist, finger, leg, or the like, of the human body,
wherein, after the first connection part of the small diameter is installed between the large bladder and the small bladder and the second connection part of the large diameter is installed on the small bladder, the second connection part of the large diameter is connected to the hose.

8. The sphygmomanometer cuff as set forth in claim 7, wherein a spacer is interposed between the large and small bladders.

9. The sphygmomanometer cuff as set forth in claim 7, wherein, when viewed in a blood flowing direction, the small bladder is positioned at the center of or at the back of the large bladder.

10. A sphygmomanometer cuff having a double bladder, comprising:
an envelope placed around a desired portion of the human body such as the arm, wrist, finger, leg, or the like and functioning to protect bladders;
a large bladder installed in the envelope to be inflated when air is supplied therein; and
a small bladder installed in the envelope in conjunction with the large bladder to be inflated when air is supplied therein and effectively apply pressure to the artery extending through the arm, wrist, finger, leg, or the like, of the human body,
wherein the large bladder is connected to the hose, and a sensor is airtightly installed in the small bladder.

11. The sphygmomanometer cuff as set forth in claim 10, wherein a spacer is interposed between the large and small bladders.

12. The sphygmomanometer cuff as set forth in claim 10, wherein, when viewed in a blood flowing direction, the small bladder is positioned at the center of or at the back of the large bladder.
